(19) United States
(12) Patent Application Publication
(10) Pub. No.: US 2017/0137762 A1
(43) Pub. Date: May 18, 2017

(54) MICROALGAE PHOTOCHEMISTRY CULTURING APPARATUS

(71) Applicant: HYUNDAI MOTOR COMPANY, Seoul (KR)

(72) Inventor: Kwang Mo SEONG, Bucheon-si (KR)

(21) Appl. No.: 15/283,866

(22) Filed: Oct. 3, 2016

(30) Foreign Application Priority Data
Nov. 12, 2015 (KR) 10-2015-0158815

(51) Int. Cl. C12M 1/00; C12M 1/107; C12M 1/12; C12M 3/06; C12M 1/26; C12M 1/34

(52) U.S. Cl. CPC C12M 21/02; C12M 33/10; C12M 47/02; C12M 41/36; C12M 25/06; C12M 29/26; C12M 27/14; C12M 21/04

(57) ABSTRACT

A microalgae photochemistry culturing apparatus includes a photochemistry culturing machine for culturing microalgae by photochemical reactions; a red variation photochemistry culturing machine, which is connected to the photochemistry culturing machine to mix a nitrogen starvation medium filled therein with the microalgae precipitated in the photochemistry culturing machine and allows the nitrogen starvation medium to subject the microalgae to red variation to produce red variation microalgae in which astaxanthin is produced; a floatation separator enriching the red variation microalgae precipitated and emitted from the red variation photochemistry culturing machine through microbubbles fed; and a centrifugal separator removing residual moisture from the red variation microalgae enriched in the floatation separator and collecting the red variation microalgae.

### CROSS-REFERENCE TO RELATED APPLICATION

This application claims under 35 U.S.C. §119(a) the benefit of priority to Korean Patent Application No. 10-2015-0158815 filed on Nov. 12, 2015, the entire content of which is incorporated herein by reference.

### TECHNICAL FIELD

The present disclosure relates to a microalgae photochemistry culturing apparatus. More particularly, the present disclosure relates to a microalgae photochemistry culturing apparatus using a microbubble apparatus.

### BACKGROUND

As global warming has recently accelerated, regulations for reducing generation and emission of greenhouse gas have been more restricted, and the greenhouse gas reduction amounts have been imposed on many countries,

Accordingly, there have been many studies to satisfy the regulations through the development of new renewable energy, energy saving technology, green cars, greenhouse gas treatment technology, and the like.

Particularly, the automotive industries have been paying attention on the greenhouse gas treatment technology together with the energy saving technology as a core technology.

The greenhouse gas treatment technology can be largely classified into a chemical treatment technology and a biological treatment technology. The chemical treatment technology includes an absorption dust capturing method using amine or potassium carbonate and an absorbent, and the biological treatment technology includes an absorption fixing method using algae which performs carbon assimilation.

Recently, the biological treatment technology has been actively studied on the absorption and fixture of carbon dioxide using microalgae. In this case, since the biological treatment technology uses living microalgae, the treatment speed is slow and a large site is required, and thus, there are limitations in securing economic efficiency.

Accordingly, there is a need for developing a culturing apparatus which may enhance the economic efficiency by culturing/collecting microalgae (Haematococcus pluvialis and Phaffia Rhodozyma) which may produce astaxanthin which has functions such as potential prevention of cancers, improvement of immune responses, and induction of antioxidant responses, and thus, is utilized as a high value-added material in the medical field.

The above information disclosed in this Background section is only for enhancement of understanding of the background of the invention, and therefore, it may contain information that does not form the prior art that is already known in this country to a person of ordinary skill in the art.

### SUMMARY OF THE DISCLOSURE

The present disclosure has been made in an effort to solve the above-described problems associated with prior art. An aspect of the present disclosure provides a microalgae photochemistry culturing apparatus which may secure economic efficiency, and thus, may secure a large-scale investment foundation by culturing and collecting microalgae containing materials for potentially preventing cancers, improving immune responses, and inducing antioxidant responses, such as astaxanthin to extract and utilize useful materials.

According to an embodiment in the present disclosure, a microalgae photochemistry culturing apparatus includes a photochemistry culturing machine for culturing microalgae by photochemical reactions, a red variation photochemistry culturing machine, which is connected to the photochemistry culturing machine to mix a nitrogen starvation medium filled therein with the microalgae precipitated in the photochemistry culturing machine and allows the nitrogen starvation medium to subject the microalgae to red variation to produce red variation microalgae in which astaxanthin is produced, a floatation separator which enriches the red variation microalgae precipitated and emitted from the red variation photochemistry culturing machine through fed microbubbles, and a centrifugal separator which removes residual moisture from the red variation microalgae enriched in the floatation separator and collects the red variation microalgae.

The red variation photochemistry culturing machine may include a culturing vessel which receives and stores a nitrogen starvation medium and microalgae, an inlet disposed at the top of the culturing vessel and into which the microalgae fed from the photochemistry culturing machine and the circulating nitrogen starvation medium are introduced, an overflow pipe inserted while having a length in the center of the culturing vessel, and an outlet connected to the overflow pipe and from which the nitrogen starvation medium mixed with microalgae is emitted.

The red variation photochemistry culturing machine may further include a microalgae emitter capable of emitting the red variation microalgae precipitated at the bottom of the culturing vessel, and the microalgae emitter may include a hopper in which the red variation microalgae grown to a predetermined weight or more are precipitated, a detection sensor capable of measuring the height of the red variation microalgae precipitated in the hopper, a top separating plate capable of separating the hopper and the culturing vessel when the red variation microalgae precipitated in the hopper are accumulated to a predetermined height or more, a bottom separating plate capable of separating the hopper and a transferring pipe of precipitated algae until the red variation microalgae precipitated in the hopper are accumulated to a predetermined height, and a separating plate driving controller which operates each of the top separating plate and the bottom separating plate by receiving signals from the detection sensor.

The red variation photochemistry culturing machine may further include a first culturing medium regenerator which regenerates the nitrogen starvation waste medium generated and fed from the culturing vessel by supplementing the waste medium with nutrients, and a first circulation pump which feeds the nitrogen starvation medium regenerated from the first culturing medium regenerator to the culturing vessel.

The red variation photochemistry culturing machine may be configured by connecting a plurality of the culturing vessels, and be disposed such that the microalgae and the nitrogen starvation medium emitted from one outlet are introduced into an inlet of another adjacent culturing vessel.

The red variation photochemistry culturing machine may have a cyclone structure to stir the nitrogen starvation medium and the microalgae and enhance an activity of the microalgae.

The microalgae photochemistry culturing apparatus may further include a microbubble generator which produces micro-bubbled water containing micronized carbon dioxide bubbles and feeds the micro-bubbled water to the floatation separator, the photochemistry culturing machine, and the red variation photochemistry culturing machine.

The microalgae photochemistry culturing apparatus may further include a second culturing medium regenerator which regenerates the waste medium generated and fed from the photochemistry culturing machine, the floatation separator, and the centrifugal separator by supplementing the waste medium with nutrients, and a second circulation pump which feeds the culturing medium regenerated from the second culturing medium regenerator to the photochemistry culturing machine.

According to the embodiment in the present disclosure, economic efficiency is secured, and thus, a large-scale investment foundation may be secured by culturing and collecting microalgae containing materials for potentially preventing cancers, improving immune responses, and inducing antioxidant responses, such as astaxanthin to extract and utilize useful materials.

In addition, by recycling a nitrogen nutrient source starvation waste medium generated from a red variation photochemistry culturing machine, a separate waste water treatment facility needs not be installed, the use of water resources and nutrients for producing a culturing medium may be decreased, and microalgae which contain the waste medium and are not grown may be utilized as they are.

Other aspects and embodiments are discussed infra.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The above and other features of the invention are discussed infra.

### BRIEF DESCRIPTION OF THE DRAWINGS

The above and other features of the present disclosure will now be described in detail with reference to certain exemplary embodiments thereof illustrated in the accompanying drawings which are given hereinbelow by way of illustration only, and thus are not limitative of the present disclosure.

FIG. 1 is a configuration view schematically illustrating a microalgae photochemistry culturing apparatus in the related art.

FIG. 2 is a configuration view schematically illustrating a microalgae photochemistry culturing apparatus according to an exemplary embodiment in the present disclosure.

FIG. 3 is a view illustrating a red variation photochemistry culturing machine of the microalgae photochemistry culturing apparatus according to an exemplary embodiment in the present disclosure.

It should be understood that the appended drawings are not necessarily to scale, presenting a somewhat simplified representation of various features illustrative of the basic principles of the invention. The specific design features of the present disclosure as disclosed herein, including, for example, specific dimensions, orientations, locations, and shapes will be determined in part by the particular intended application and use environment.

In the figures, reference numbers refer to the same or equivalent parts of the present disclosure throughout the several figures of the drawing.

### DETAILED DESCRIPTION

Hereinafter reference will now be made in detail to various embodiments in the present disclosure, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents, and other embodiments, which may be included within the spirit and scope of the invention as defined by the appended claims.

Hereinafter, exemplary embodiments according to the present disclosure will be described in detail with reference to the accompanying drawings.

The benefits and features of the present disclosure and the methods of achieving the benefits and features will become apparent with reference to the exemplary embodiments to be described below in detail along with the accompanying drawings.

However, the present disclosure is not limited to the exemplary embodiments to be disclosed below, but may be implemented in various other forms, and the present exemplary embodiments are provided for rendering the disclosure of the present disclosure complete and for fully representing the scope of the invention to a person with ordinary skill in the technical field to which the present disclosure pertains, and the present disclosure will be defined only by the scope of the claims.

The known technology and the like may obscure the gist of the present disclosure in describing the present disclosure, and thus, the detailed description thereof will be omitted.

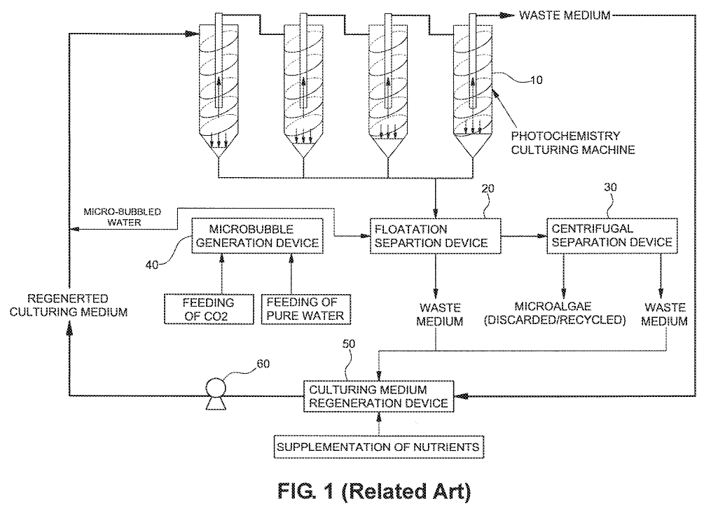

FIG. 1 is a configuration view schematically illustrating a microalgae photochemistry culturing apparatus in the related art.

As illustrated in FIG. 1, the microalgae photochemistry culturing apparatus in the related art includes; a photochemistry culturing machine 10 in the form of a cyclone; a floatation separation device 20 for enriching microalgae precipitated; a centrifugal separation device 30 for collecting the enriched microalgae; a microbubble generation device 40 for producing microbubbles with a diameter of approximately 30 μm using carbon dioxide; a culturing medium regeneration device 50 for preparing a regenerated culturing medium by supplementing a waste medium with nutrients; and a circulation pump 60. The efficiency of treating carbon dioxide may be improved.

The microalgae photochemistry culturing apparatus in the related art may enhance the efficiency of treating carbon dioxide gas by using a microbubble generator to generate μm-sized carbon dioxide microbubbles and utilize the microbubbles, and a device such as a separate compressor for feeding carbon dioxide need not be provided. Thus, energy costs may be reduced, and simultaneously, problems related to expenses/spaces may be solved.

The microalgae photochemistry culturing apparatus in the related art has an additional effect in that the amount of nutrients used may be reduced by recycling a waste medium, water resources required for culturing may be saved, and waste water treatment facilities for treating the waste medium may be omitted.

The microalgae collected through the centrifugal separation device 30 of the microalgae photochemistry culturing apparatus in the related art may be discarded or used as a raw material for biodegradable plastic or bio fuel.

However, when microalgae are discarded, costs for treatment are required. When microalgae are recycled as biodegradable plastic and bio fuel, there is a problem in that insufficient demands for those materials reduce the economic efficiency.

Since the problem may also serve as a barrier for investing in large-scale treatment facilities for treating carbon dioxide (CO₂), the present disclosure is intended to overcome the aforementioned problems by culturing a large amount of microalgae containing astaxanthin, which has been recently utilized as a high value-added material in the medical field to extract and utilize astaxanthin, thereby securing economic efficiency.

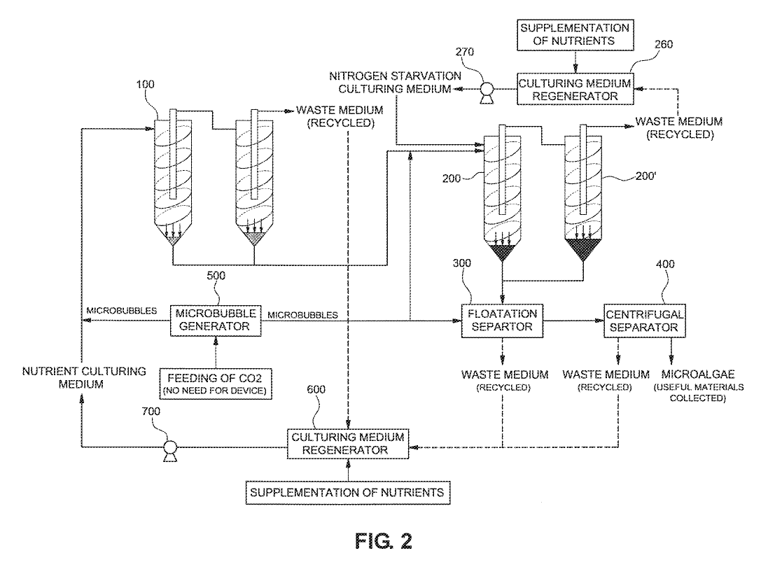

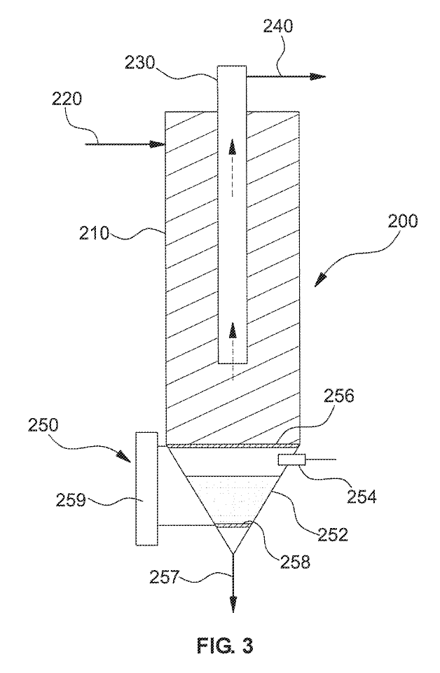

Hereinafter, FIG. 2 is a configuration view schematically illustrating a microalgae photochemistry culturing apparatus according to an exemplary embodiment in the present disclosure, and FIG. 3 is a view illustrating a red variation photochemistry culturing machine of a microalgae photochemistry culturing apparatus according to an exemplary embodiment in the present disclosure.

As illustrated in FIG. 2, the microalgae photochemistry culturing apparatus has been created based on that microalgae capable of producing astaxanthin increase the production of astaxanthin in a state where nitrogen nutrient sources lack, and the apparatus includes a photochemistry culturing machine 100, a red variation photochemistry culturing machine 200, a floatation separator 300, and a centrifugal separator 400 for this purpose.

The photochemistry culturing machine 100 cultures microalgae by photochemical reactions, and is the same as the photochemistry culturing machine 10 in the related art in terms of the shape and characteristics.

The red variation photochemistry culturing machine 200 has the same structure and configuration as those of the photochemistry culturing machine 100, and is connected to the photochemistry culturing machine 100 to mix a nitrogen starvation medium filled therein with the microalgae precipitated in the photochemistry culturing machine 100.

The red variation photochemistry culturing machine 200 allows the nitrogen starvation medium to subject the microalgae to red variation, thereby producing red variation microalgae in which astaxanthin is produced.

That is, the red variation photochemistry culturing machine 200 allows the microalgae grown and precipitated in the photochemistry culturing machine 100 to be transferred and subjected to red variation while growing on a nitrogen starvation medium in which nitrogen nutrient sources lack, thereby producing astaxanthin.

The red variation photochemistry culturing machine 200 includes a culturing vessel 210, an inlet 220, an overflow pipe 230, and an outlet 240, as illustrated in FIG. 3.

In other words, the red variation photochemistry culturing machine 200 has the form of a cyclone including the culturing vessel 210 capable of receiving and storing the culturing medium and the microalgae, the inlet 220 and the outlet 240, which are disposed at the top of the culturing vessel 210, and an overflow pipe 230 installed in connection to the outlet 240 to have a predetermined length in the center of the culturing vessel 210 and longitudinally inserted.

Here, into the inlet 220, a nitrogen starvation medium fed by a first circulation pump 270 and the microalgae transferred from the photochemistry culturing machine 100 or a nitrogen starvation waste medium emitted from the outlet 240 of another culturing vessel 210 are or is introduced, and from the outlet 240, a culturing medium mixed with microalgae which have not been grown is emitted.

The red variation photochemistry culturing machine 200 is disposed while having a cyclone structure, and accordingly, when the nitrogen starvation medium is fed to the inlet 220 by the first circulation pump 270, a vortex is formed inside the culturing vessel 210 by the cyclone principle according to the shape. As a result, the red variation microalgae grown to a predetermined weight or more are precipitated at the bottom of the culturing vessel 210, and the residual microalgae are emitted together with the nitrogen starvation waste medium through the overflow pipe 230 to the outlet 240.

The microalgae photochemistry culturing apparatus according to the present disclosure may connect a plurality of the red variation photochemistry culturing machines 200 having the cyclone form as illustrated in FIG. 3 in separation from the photochemistry culturing machine 100, and accordingly, is disposed such that the microalgae and the nitrogen starvation waste medium emitted from one outlet 240 are introduced into an outlet 220 of another adjacent culturing vessel.

More specifically, the red variation photochemistry culturing machines 200 have the outlet 240 of any one red variation photochemistry culturing machine 200 is connected to the inlet 220 of another red variation photochemistry culturing machine 200′ to introduce the nitrogen starvation waste medium emitted from the outlet 240 of one red variation photochemistry culturing machine 200 into the inlet 220 of another red variation photochemistry culturing machine 200′.

Accordingly, the nitrogen starvation waste medium (including microalgae which have not been grown) emitted to outside of the culturing vessel 210 through the overflow pipe 230 is introduced into another connected red variation photochemistry culturing machine 200′.

As illustrated in FIG. 3, the red variation photochemistry culturing machine further includes a microalgae emitter 250 capable of emitting the red variation microalgae precipitated at the bottom of the culturing vessel 210.

The microalgae emitter 250 as described above includes: a hopper 252 in which the red variation microalgae grown to a predetermined weight or more are naturally precipitated and accumulated; a detection sensor 254 measuring the height of the red variation microalgae precipitated in the hopper 252; a top separating plate 256 capable of separating the hopper 252 and the culturing vessel 210 when the red variation microalgae precipitated in the hopper are accumulated to a predetermined height or more; a bottom separating plate 258 which separates the hopper 252 and a transferring pipe 257 of precipitated algae until the red variation microalgae precipitated in the hopper 252 are accumulated to a predetermined height; and a separating plate driving controller 259 which operates each of the top separating plate 256 and the bottom separating plate 258 by receiving signals from the detection sensor 254.

Here, the top separating plate 256 is usually maintained in a folded state to provide a channel through which the red variation microalgae may be precipitated from the culturing vessel 210 to the hopper 252, and is unfolded for the emission of the microalgae to separate the hopper 252 and the culturing vessel 210 when the red variation microalgae precipitated in the hopper 252 are accumulated to a predetermined height or more.

The bottom separating plate 258 is usually maintained in an unfolded state to separate the hopper 252 and the transferring pipe 257 of precipitated algae, and is folded for the emission of the red variation microalgae to provide a channel through which the microalgae accumulated in the hopper 252 may be emitted to the transferring pipe 257 of precipitated algae when the red variation microalgae precipitated in the hopper 252 are accumulated to a predetermined height or more.

The transferring pipe 257 of precipitated algae is connected to the lower end of the hopper 252, and transfers the red variation microalgae emitted from the hopper 252 to the floatation separator 300 when the bottom separating plate 258 is folded.

The microalgae photochemistry culturing apparatus according to the present disclosure may further include a first culturing medium regenerator 260 and a first circulation pump 270.

The first culturing medium regenerator 260 regenerates the nitrogen starvation waste medium generated and transferred from the culturing vessel 210 by supplementing the waste medium with nutrients.

The first circulation pump 270 feeds the nitrogen starvation medium regenerated by the first culturing medium regenerator 260 to the culturing vessel 210.

Accordingly, since the nitrogen starvation medium may be continuously fed to the red variation photochemistry culturing machine 200 by regenerating the waste medium emitted from the red variation photochemistry culturing machine 200 in the present disclosure, it is possible to effectively increase the production of the red variation microalgae in which astaxanthin is produced from the red variation photochemistry culturing machine 200.

The floatation separator 300 according to the present disclosure enriches the red variation microalgae precipitated and emitted from the red variation photochemistry culturing machine through microbubbles fed by a microbubble generator 500.

That is, the floatation separator 300 produces micro-bubbled water containing micronized carbon dioxide bubbles, When the red variation microalgae are floated by carbon dioxide microbubbles fed by the microbubble generator 500 which feeds the micro-bubbled water to the floatation separator 300, the photochemistry culturing machine 100, and the red variation photochemistry culturing machine 200, the red variation microalgae are removed by a skimmer and enriched and stored in a microalgae storage bath (not illustrated).

The red variation microalgae enriched in the microalgae storage bath (not illustrated) are transferred to the centrifugal separator 400, and the waste medium generated during the process of enriching the red variation microalgae is transferred to a second culturing medium regenerator 600.

The centrifugal separator 400 removes and collects residual moisture of the red variation microalgae enriched in the floatation separator 300, and may be composed of a typical centrifugal separator. The red variation microalgae collected from the centrifugal separator 400 are transferred to a facility for extracting astaxanthin. The waste medium generated during the process is transferred to the second culturing medium regenerator 600 in the same manner as in the floatation separator 300, and then, is regenerated and recycled for culturing microalgae.

Here, the second culturing medium regenerator 600 regenerates the waste medium generated and transferred from the photochemistry culturing machine 100, the floatation separator 300, and the centrifugal separator 400 by supplementing the waste medium with nutrients. The regenerated culturing medium is formed so as to be fed to the photochemistry culturing machine 100 through a second circulation pump 700.

According to the present disclosure, economic efficiency is secured, and thus, a large-scale investment foundation may be secured by culturing and collecting microalgae containing materials for potentially preventing cancers, improving immune responses, and inducing antioxidant responses, such as astaxanthin to extract and utilize useful materials.

The present disclosure also has effects in that by recycling nitrogen nutrient source starvation waste medium generated from a red variation photochemistry culturing machine, a separate waste water treatment facility needs not be installed, the use of water resources and nutrients for producing a culturing medium may be decreased, and microalgae which contain the waste medium and are not grown may be utilized as they are.

The invention has been described in detail with reference to exemplary embodiments thereof. However, it will be appreciated by those skilled in the art that changes may be made in these embodiments without departing from the principles and spirit of the invention, the scope of which is defined in the appended claims and their equivalents.

What is claimed is:

1. A microalgae photochemistry culturing apparatus comprising:
a photochemistry culturing machine for culturing microalgae by photochemical reactions;
a red variation photochemistry culturing machine connected to the photochemistry culturing machine to mix a nitrogen starvation medium filled therein with the microalgae precipitated in the photochemistry culturing machine, the red variation photochemistry culturing machine allowing the nitrogen starvation medium to subject the microalgae to red variation to produce red variation microalgae in which astaxanthin is produced;
a floatation separator enriching the red variation microalgae precipitated and emitted from the red variation photochemistry culturing machine through microbubbles fed; and
a centrifugal separator removing residual moisture from the red variation microalgae enriched in the floatation separator and collecting the red variation microalgae.

2. The microalgae photochemistry culturing apparatus of claim 1, wherein the red variation photochemistry culturing machine comprises:
a culturing vessel receiving and storing the nitrogen starvation medium and microalgae;
an inlet disposed at a top of the culturing vessel and into which the microalgae fed from the photochemistry culturing machine and the circulating nitrogen starvation medium are introduced;
an overflow pipe inserted while having a length in the center of the culturing vessel; and
an outlet connected to the overflow pipe and from which the nitrogen starvation medium mixed with microalgae is emitted.

3. The microalgae photochemistry culturing apparatus of claim 2, wherein the red variation photochemistry culturing machine further comprises:
a microalgae emitter emitting the red variation microalgae precipitated at a bottom of the culturing vessel,
wherein the microalgae emitter includes:
a hopper in which the red variation microalgae grown to a reference weight or more are precipitated;
a detection sensor measuring the height of the red variation microalgae precipitated in the hopper;
a top separating plate separating the hopper and the culturing vessel when the red variation microalgae precipitated in the hopper are accumulated to the reference height or more;
a bottom separating plate separating the hopper and a transferring pipe of precipitated algae until the red variation microalgae precipitated in the hopper are accumulated to the reference height; and
a separating plate driving controller configured to operate each of the top separating plate and the bottom separating plate by receiving signals from the detection sensor.

4. The microalgae photochemistry culturing apparatus of claim 2, wherein the red variation photochemistry culturing machine further comprises:
a first culturing medium regenerator regenerating the nitrogen starvation waste medium, which is generated and fed from the culturing vessel, by supplementing the waste medium with nutrients; and
a first circulation pump feeding the nitrogen starvation medium, which is regenerated from the first culturing medium regenerator, to the culturing vessel.

5. The microalgae photochemistry culturing apparatus of claim 2, wherein the red variation photochemistry culturing machine includes a plurality of culturing vessels and is disposed such that the microalgae and the nitrogen starvation medium emitted from one outlet are introduced into an inlet of another adjacent culturing vessel.

6. The microalgae photochemistry culturing apparatus of claim 1, wherein the red variation photochemistry culturing machine has a cyclone structure to stir the nitrogen starvation medium and the microalgae and to enhance activity of the microalgae.

7. The microalgae photochemistry culturing apparatus of claim 1, further comprising:
a micro bubble generator producing micro-bubbled water, which contains micronized carbon dioxide bubbles, and feeding the micro-bubbled water to the floatation separator, the photochemistry culturing machine, and the red variation photochemistry culturing machine.

8. The microalgae photochemistry culturing apparatus of claim 1, further comprising:
a second culturing medium regenerator regenerating the waste medium, which is generated and fed from the photochemistry culturing machine, the floatation separator, and the centrifugal separator, by supplementing the waste medium with nutrients; and
a second circulation pump feeding the culturing medium, which is regenerated from the second culturing medium regenerator, to the photochemistry culturing machine.
